(19) United States
(12) Patent Application Publication
(10) Pub. No.: US 2013/0252277 A1
(43) Pub. Date: Sep. 26, 2013

(54) METHOD OF OPERATION OF FERMENTATION OF CARBON MONOXIDE CONTAINING GASEOUS SUBSTRATE

(71) Applicant: INEOS BIO SA, Lisle, IL (US)

(72) Inventors: Ryan Senaratne, Fayetteville, AR (US); Brandon Beard, Springdale, AR (US)

(21) Appl. No.: 13/989,111

(22) PCT Filed: Nov. 14, 2011
(86) PCT No.: PCT/US2011/001900

§ 371 Date: May 23, 2013

Related U.S. Application Data
(60) Provisional application No. 61/458,903, filed on Dec. 3, 2010.

(51) Int. Cl. C12P 7/54; C12P 7/06; C12P 7/16

(52) U.S. Cl. CPC C12P 7/54; C12P 7/16; C12P 7/065
USPC 435/39; 435/160; 435/161; 435/140

(57) ABSTRACT

The present disclosure provides methods of gaseous substrate fermentation comprising: adding gaseous substrate into an aqueous medium in a bioreactor. The methods of the present disclosure comprise: measuring cell density; adjusting input of gaseous substrate to increase cell density; changing specific CO uptake in predetermined amounts.

The present disclosure comprises fermentation methods of a gaseous substrate comprising carbon monoxide. The present disclosure provides fermentation methods of a gaseous substrate comprising carbon monoxide to produce one or more alcohols.

### BACKGROUND

Methods for producing chemicals such as organic acids, e.g. acetic acid and alcohols, e.g. ethanol from microbial fermentation of gaseous substrates comprising carbon monoxide in media containing suitable nutrients and trace minerals using certain microorganisms, such as those from the genus Clostridium, have been demonstrated. For example, U.S. Pat. No. 5,173,429 to Gaddy et al. discloses Clostridium ljungdahlii ATCC No. 49587, an anaerobic microorganism that produces ethanol and acetate from synthesis gas. U.S. Pat. No. 5,807,722 to Gaddy et al. discloses a method and apparatus for converting waste gases into useful products such as organic acids and alcohols using anaerobic bacteria, such as Clostridium ljungdahlii ATCC No. 55380. U.S. Pat. No. 6,136,577 to Gaddy et al. discloses a method and apparatus for converting waste gases into useful products such as organic acids and alcohols (particularly ethanol) using anaerobic bacteria, such as Clostridium ljungdahlii ATCC Nos. 55988 and 55989.

US Patent Application No. 20070275447 discloses a clostridium bacterial species (Clostridium carboxidivorans, ATCC BAA-624, “P7”) that is capable of synthesizing, from waste gases, products which are useful as biofuel. U.S. Pat. No. 7,704,723 discloses a clostridium bacterial species (Clostridium ragsdalei, ATCC BAA-622, “P11”) that is capable of synthesizing, from waste gases, products which are useful as biofuel.

WO 2007/117157 discloses use of Clostridium autoethanogenum (Accession No. DSM 10061, DSMZ, Germany) for the production of ethanol by anaerobic fermentation of substrates containing carbon monoxide. WO 2009/064200 discloses another bacteria (Clostridium autoethanogenum, Accession No. DSM 19630, DSMZ, Germany) for the production of ethanol by anaerobic fermentation of substrates containing carbon monoxide.

As described in the art, rate of production of chemicals, such as alcohol, depend on cell density in the fermentation medium. Proper high cell density in the bioreactor is required in order to attain and maintain a high rate of production of chemicals.

U.S. Pat. No. 6,136,577 to Gaddy discloses a process of ethanol production in a fermentation process wherein cell-recycle is used to increase cell density.

U.S. Pat. No. 7,285,402 to Gaddy et al. discloses an anaerobic microbial fermentation process for the production of alcohol wherein a method of increasing cell density is presented during start up using a stock culture wherein there was excess H₂ present.

Start-up using a batch inoculum from stock culture ensures a healthy inoculum free from contaminants, but is not always successful as an inoculation procedure because of the rather low cell density employed, especially if the method parameters such as gas rate and agitation rate are pushed upward too rapidly just after inoculation.

Currently, there is a need in the art for improved methods to increase cell density in microbial fermentation of a gaseous substrate. The present disclosure provides a method to increase cell density at a faster rate for microbial fermentation methods of a gaseous substrate.

### SUMMARY

The present disclosure provides a process of producing one or more alcohols from a gaseous substrate, comprising: fermenting a gaseous substrate comprising carbon monoxide (CO) into an aqueous medium in a bioreactor; said process comprising increasing cell density by adjusting specific CO uptake in said aqueous medium; wherein rate of change of specific CO uptake comprises controlling rate of input of CO; wherein rate of change of specific CO uptake comprises measuring rate of CO input; measuring rate of CO output; and measuring cell mass; wherein further comprising changing specific CO uptake in predetermined amounts; wherein the said predetermined amounts comprise a range of about 0.001 to about 10.0 mmol/min/gram dry cell; wherein the said predetermined amounts comprise a range of about 0.01 to about 5.0 mmol/min/gram dry cell; wherein the said predetermined amounts comprise a range of about 0.1 to about 1.0 mmol/min/gram dry cell; further comprising adding a flow of aqueous medium into bioreactor; removing a flow of fermentation broth from the bioreactor; further comprising adding a continuous flow of aqueous medium into bioreactor; removing a continuous flow of fermentation broth from the bioreactor; wherein said aqueous medium comprises one or more of the microorganism including: biologically pure microorganism, naturally occurring microorganism, non-naturally occurring microorganism, non-naturally occurring microorganism produced by genetic modification, mutant of naturally occurring microorganism, mutant of non-naturally occurring microorganism, recombinant microorganism, engineered microorganism, artificially synthesized microorganism; wherein said bioreactor comprises one or more reactor; wherein said bioreactor comprises cell recycle unit; wherein said CO-containing substrate comprises hydrogen; further comprising adding nutrient medium to said bioreactor.

As an embodiment, the present disclosure provides a method gaseous substrate fermentation comprising: adding gaseous substrate comprising carbon monoxide into an aqueous medium in a bioreactor; said method comprising selecting target specific CO uptake; adjusting flow of gaseous substrate such that specific CO uptake equals said target specific CO uptake in a range of about 0.01 to about 10 mmol/min/gram dry cell.

As an embodiment, the present disclosure provides a method gaseous substrate fermentation comprising: adding gaseous substrate comprising carbon monoxide into an aqueous medium in a bioreactor; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.

As an embodiment, the present disclosure provides a method gaseous substrate fermentation comprising: adding gaseous substrate comprising carbon monoxide into an aqueous medium in a bioreactor; said method comprising selecting target specific CO uptake; adjusting flow of gaseous substrate such that specific CO uptake equals said target specific CO uptake in a range of about 0.01 to about 10 mmol/min/gram dry cell.

As an embodiment, the present disclosure provides a method gaseous substrate fermentation comprising: adding gaseous substrate comprising carbon monoxide into an aqueous medium in a bioreactor; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.

The present disclosure provides a method of gaseous substrate fermentation comprising: adding gaseous substrate comprising carbon monoxide (CO) into an aqueous medium in a bioreactor; said aqueous medium comprising one or more microorganism; said method comprising increasing cell density by adjusting specific CO uptake of one or more said microorganism in said aqueous medium; in one embodiment, rate of change of specific CO uptake comprises adjusting one or more steps; in one embodiment, rate of change of specific CO uptake comprises controlling rate of input of CO; in one embodiment, rate of change of specific CO uptake comprises measuring rate of CO input; measuring rate of CO output; and measuring cell mass; further comprises changing specific CO uptake in predetermined amounts; in one embodiment, the said predetermined amounts comprise a range of about 0.001 to about 10.0 mmol/min/gram dry cell; in one embodiment, the said predetermined amounts comprise a range of about 0.01 to about 5.0 mmol/min/gram dry cell; in one embodiment, the said predetermined amounts comprise a range of about 0.1 to about 1.0 mmol/min/gram dry cell; in one embodiment, adding continuous flow of aqueous medium into bioreactor; removing a continuous flow of fermentation broth from the bioreactor; wherein repeating said steps until obtaining a desired ethanol productivity a range of about 1 to about 50 g/L; in one embodiment, adding continuous flow of aqueous medium into bioreactor; removing a continuous flow of fermentation broth from the bioreactor; wherein repeating said steps until obtaining a desired ethanol concentration in the fermentation broth a range of about 1 to about 50 g/L.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising selecting target specific CO uptake; adjusting flow of gaseous substrate such that specific CO uptake equals said target specific CO uptake in a range of about 0.01 to about 10 mmol/min/gram dry cell.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.

The present disclosure provides a method of gaseous substrate fermentation comprising: adding gaseous substrate comprising carbon monoxide into an aqueous medium in a bioreactor; said aqueous medium comprising one or more microorganism; said method comprising measuring cell density; adjusting input of gaseous substrate to increase cell density; changing specific CO uptake in predetermined amounts in a range of about 0.001 to about 10.0 mmol/min/gram dry cell.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising selecting target specific CO uptake; adjusting flow of gaseous substrate such that specific CO uptake equals said target specific CO uptake in a range of about 0.01 to about 10 mmol/min/gram dry cell.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.

As embodiments the method of the present disclosure wherein: said microorganism comprises one or more of biologically pure anaerobic acetogenic bacteria; wherein said microorganism comprises one or more of naturally occurring anaerobic acetogenic bacteria; wherein said microorganism comprises one or more of non-naturally occurring anaerobic acetogenic bacteria; wherein said microorganism comprises one or more of non-naturally occurring anaerobic acetogenic bacteria produced by genetic modification using anaerobic acetogenic bacteria as host organism; wherein said microorganism comprises one or more of non-naturally occurring anaerobic acetogenic bacteria produced by inserting genes of anaerobic acetogenic bacteria into a host organism.

As an embodiment, said microorganism of the present disclosure comprises one or more of the microorganism including: biologically pure microorganism, naturally occurring microorganism, non-naturally occurring microorganism, non-naturally occurring microorganism produced by genetic modification, mutant of naturally occurring microorganism, mutant of non-naturally occurring microorganism, recombinant microorganism, engineered microorganism, artificially synthesized microorganism; wherein said microorganism comprises selection from Acetogenium kivui, Acetobacterium woodii, Acetoanaerobium noterae, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium autoethanogenum (DSM 23693), Clostridium autoethanogenum (DSM 19630 of DSMZ Germany), Clostridium autoethanogenum (DSM 10061 of DSMZ Germany), Clostridium thermoaceticum, Eubacterium limosum, Clostridium ljungdahlii PETC (ATCC 49587), Clostridium ljungdahlii ER12 (ATCC 55380), Clostridium ljungdahlii C-01 (ATCC 55988), Clostridium ljungdahlii O-52 (ATCC 55889), Clostridium ultunense, Clostridium ragsdali P11 (ATCC BAA-622), Alkalibaculum bacchi CP11 (ATCC BAA-1772), Clostridium coskatii, Clostridium carboxidivorans P7 (ATCC PTA-7827), Geobacter sulfurreducens, Morrella thermacetica, Peptostreptococcus productus, Clostridium drakei, recombinant microorganism (DSM 24138), and mixtures thereof.

As an embodiment the method of the present disclosure wherein said microorganism comprises one or more strains of Clostridium ljundahlii, or one or more strains of Clostridium ragsdalei, or one or more strains of Clostridium carboxidivorans, or one or more strains of Clostridium autoethanogenum.

As an embodiment the method of the present disclosure wherein said microorganism comprises one or more genetically modified microorganism produced by inserting one or more selected genes into host organism selected from any Clostridium ljundahlii strains, or any Clostridium ragsdalei strains, or any Clostridium carboxidivorans strains, or any Clostridium autoethanogenum strains.

As an embodiment the method of the present disclosure wherein said microorganism comprises one or more genetically modified microorganism produced by inserting into any host organism one or more genes from any Clostridium ljundahlii strain, or any Clostridium ragsdalei strain, or any Clostridium carboxidivorans strain, or any Clostridium autoethanogenum strain.

As embodiments the method of the present disclosure: wherein said bioreactor comprises one or more reactor; wherein said bioreactor comprises cell recycle unit; wherein said CO-containing substrate comprises hydrogen.

As an embodiment, further comprising adding nutrient medium to said bioreactor.

### DESCRIPTION OF FIGURES

FIG. 1 comprises a schematic diagram illustrating an embodiment of the process of microbial fermentation of a gaseous substrate.

### DEFINITIONS

Unless otherwise defined, the following terms as used throughout this specification for the present disclosure are defined as follows and can include either the singular or plural forms of definitions below defined:

The term “about” modifying any amount refers to the variation in that amount encountered in real world conditions of sustaining microorganism culture, e.g., in the lab, pilot plant, or production facility. For example, an amount of an ingredient or measurement employed in a mixture or quantity when modified by “about” includes the variation and degree of care typically employed in measuring in an experimental condition in production plant or lab. For example, the amount of a component of a product when modified by “about” includes the variation between batches in a multiple experiments in the plant or lab and the variation inherent in the analytical method. Whether or not modified by “about,” the amounts include equivalents to those amounts. Any quantity stated herein and modified by “about” can also be employed in the present disclosure as the amount not modified by “about.”

The term “acetogen” or “acetogenic” refers to a bacterium that generates acetate as a product of anaerobic respiration. These organisms are also referred to as acetogenic bacteria, since all known acetogens are bacteria. Acetogens are found in a variety of habitats, generally those that are anaerobic (lack oxygen). Acetogens can use a variety of compounds as sources of energy and carbon; the best studied form of acetogenic metabolism involves the use of carbon dioxide as a carbon source and hydrogen as an energy source.

The terms “bioreactor,” “reactor,” or “fermentation bioreactor,” include a fermentation device consisting of one or more vessels and/or towers or piping arrangement, which includes the Continuous Stirred Tank Reactor (CSTR), Bubble Column, Gas lift Fermenter, Static Mixer, or other device suitable for gas-liquid contact. For the method of this disclosure, the fermentation bioreactor may comprise a growth reactor which feeds the fermentation broth to a second fermentation bioreactor, in which most of the product, ethanol, is produced.

The term “cell density” means mass of microorganism cells per unit volume of fermentation broth, e.g. g/liter.

The term “cell recycle” means arrangement of separating liquid (permeate) from solid microorganism cells in a fermentation broth and returning all or part of said separated solid microorganism cells back to fermentor that produced said fermentation broth using said microorganism. Generally a filtration device is used to accomplish said separation. A stream of solid microorganism free permeate stream and a stream of concentrated solid microorganism is produced from the filtration device. The solid free permeate stream may contain solid particles less than a specified particle size.

The term “conversion” means a fraction of input quantity that is converted into product(s); this is denoted in the following equation: (input quantity−output quantity)/(input quantity).

The term “ethanol productivity” means amount of ethanol produced per unit fermentor volume per day. The fermentor volume is effective volume or liquid volume in the fermentor.

The term “fermentation” means fermentation of CO to alcohols and acetate. A number of bacteria are known to be capable of carrying out the fermentation of CO to alcohols, including butanol and ethanol, and acetic acid, and are suitable for use in the process of the present disclosure. Examples of such bacteria that are suitable for use in the disclosure include those of the genus Clostridium, such as strains of Clostridium ljungdahlii, including those described in WO 2000/68407, EP 117309, U.S. Pat. Nos. 5,173,429, 5,593,886, and 6,368,819, WO 1998/00558 and WO 2002/08438, strains of Clostridium autoethanogenum (DSM 10061 and DSM 19630 of DSMZ, Germany) including those described in WO 2007/117157 & WO 2009/151342 and Clostridium ragsdalei (P11, ATCC BAA-622) including those described respectively in U.S. Pat. No. 7,704,723 and “Biofuels and Bioproducts from Biomass—Generated Synthesis Gas,” Hasan Atiyeh, presented in Oklahoma EPSCoR Annual State Conference, Apr. 29, 2010 and Clostridium carboxidivorans (ATCC BAA-624) described in US Patent Application No. 20070275447. Other suitable bacteria include those of the genus Moorella, including Moorella sp HUC22-1, and those of the genus Carboxydothermus. The disclosures of each of these publications are fully incorporated herein by reference. In addition, other bacteria may be selected for use in the process of the disclosure by a person of skill in the art. It will also be appreciated that a mixed culture of two or more bacteria may be used in the process of the present disclosure. One microorganism suitable for use in the present disclosure is Clostridium autoethanogenum. Fermentation may be carried out in any suitable bioreactor, such as a continuous stirred tank reactor (CTSR), a bubble column reactor (BCR) or a trickle bed reactor (TBR). Also, in some preferred embodiments of the disclosure, the bioreactor may comprise a first, growth reactor in which the microorganisms are cultured, and a second, fermentation reactor, to which fermentation broth from the growth reactor is fed and in which most of the fermentation product (ethanol and acetate) is produced.

The term “fermentation broth” means: the composition of the fermentation medium comprises anything that ends up in the fermentation broth including: raw substrates, fermentation products, microorganism(s) and derived components, chemical additives, nutrients, gases. All three main phases; solid, liquid and gases are present in the fermentation broth and their possible interactions

The term “gene” means a segment of DNA; it may include regions preceding and following the coding DNA as well as introns between the exons; may be a unit of heredity; in this disclosure the term “gene” includes a DNA segment that contributes to phenotype/function; the segments of DNA which cells transcribe into RNA and translate, at least in part, into proteins; a sequence (a string) of bases made up of combinations of A, T, C, and G. Generally, as provided in this disclosure, the definition can refer to either singular or plural meanings.

The term “microorganism” or “microbe” includes bacteria, fungi, yeast, archaea, and protists; microscopic plants (called green algae); and animals such as plankton, the planarian and the amoeba. Some also include viruses, but others consider these as non-living. Microorganisms live in all parts of the biosphere where there is liquid water, including soil, hot springs, on the ocean floor, high in the atmosphere and deep inside rocks within the Earth's crust. Microorganisms are critical to nutrient recycling in ecosystems as they act as decomposers. Microbes are also exploited by people in biotechnology, both in traditional food and beverage preparation, and in modern technologies based on genetic engineering. It is envisioned that mixed strain microorganisms, that may or may not contain strains of various microorganisms, will be utilized in the present disclosure. Also, it is envisioned that directed evolution can selectively screen microorganisms that can be utilized in the present disclosure. It is further envisioned that recombinant DNA technology can create microorganisms using select strains of existing microorganisms. Also chemical mutagenesis technology (mutating bacterial DNA using various chemicals) can create microorganisms using select strains of existing microorganisms. It is envisioned that bacteria which are able to convert CO and water or H₂ and CO₂ into ethanol and acetic acid products will be utilized in the present disclosure. Some examples of useful bacteria include Acetogenium kivui, Acetobacterium woodii, Acetoanaerobium noterae, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium autoethanogenum (DSM 23693), Clostridium autoethanogenum (DSM 19630 of DSMZ Germany), Clostridium autoethanogenum (DSM 10061 of DSMZ Germany), Clostridium thermoaceticum, Eubacterium limosum, Clostridium ljungdahlii PETC (ATCC 49587), Clostridium ljungdahlii ER12 (ATCC 55380), Clostridium ljungdahlii C-01 (ATCC 55988), Clostridium ljungdahlii O-52 (ATCC 55889), Clostridium ultunense, Clostridium ragsdali P11 (ATCC BAA-622), Alkalibaculum bacchi CP11 (ATCC BAA-1772), Clostridium coskatii, Clostridium carboxidivorans P7 (ATCC PTA-7827), Geobacter sulfurreducens, Morrella thermacetica, Peptostreptococcus productus, Clostridium drakei, recombinant microorganism (DSM 24138), and mixtures thereof. Other bacteria may be selected for use in these methods by one of skill in the art. Generally, as provided in this disclosure, the definition can refer to either singular or plural meanings.

The term “nutrient medium” comprises microorganism growth medium which may contain one or more of vitamins and minerals that permit growth of selected microorganism. Components of a variety of nutrient media suitable to the use of this invention are known and reported in prior publications such as International Patent Application No. WO 2008/00558, U.S. Pat. No. 7,285,402, U.S. Pat. No. 5,807,722; U.S. Pat. No. 5,593,886, and U.S. Pat. No. 5,821,111.

The term “specific CO uptake” means amount of CO in m-moles consumed by unit mass of microorganism cells (g) per unit time in min, i.e. m-mole/g/min.

The term “substrate” means a substance that is acted upon by an enzyme or microorganism to produce fermentation product. For example, sugar in sugar fermentation by enzymes to produce ethanol, one or more of CO, CO2 and H2 in syngas fermentation by microorganism to produce one or more of carboxylic acid and alcohol.

The term “syngas” or “synthesis gas” means synthesis gas which is the name given to a gas mixture that contains varying amounts of carbon monoxide and hydrogen. Examples of production methods include steam reforming of natural gas or hydrocarbons to produce hydrogen, the gasification of coal and in some types of waste-to-energy gasification facilities. The name comes from their use as intermediates in creating synthetic natural gas (SNG) and for producing ammonia or methanol. Syngas is also used as intermediate in producing synthetic petroleum for use as a fuel or lubricant via Fischer-Tropsch synthesis and previously the Mobil methanol to gasoline process. Syngas consists primarily of hydrogen, carbon monoxide, and very often some carbon dioxide.

### DETAILED DESCRIPTION

The present disclosure provides a method of gaseous substrate fermentation comprising: adding gaseous substrate comprising carbon monoxide (CO) into an aqueous medium in a bioreactor; said aqueous medium comprising one or more microorganism; said method comprising increasing cell density by adjusting specific CO uptake of one or more said microorganism in said aqueous medium; wherein rate of change of specific CO uptake comprises adjusting and/or controlling one or more of the following steps: controlling rate of input of CO; measuring rate of CO input; measuring rate of CO output; measuring cell mass; further comprises changing specific CO uptake in predetermined amounts; wherein the said predetermined amounts comprise a range of about 0.001 to about 10.0 mmol/min/gram dry cell; optionally in one embodiment, said predetermined amounts comprise a range of about 0.01 to about 5.0 mmol/min/gram dry cell and/or a range of about 0.1 to about 1.0 mmol/min/gram dry cell; one embodiment optionally comprises adding continuous flow of aqueous medium into bioreactor; removing a continuous flow of fermentation broth from the bioreactor; wherein repeating said steps until obtaining a desired ethanol productivity a range of about 1 to about 50 g/L; one embodiment, optionally comprises adding continuous flow of aqueous medium into bioreactor; removing a flow of fermentation broth from the bioreactor; wherein repeating said steps until obtaining a desired ethanol concentration in the fermentation broth a range of about 1 to about 50 g/L.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising selecting target specific CO uptake; adjusting flow of gaseous substrate such that specific CO uptake equals said target specific CO uptake in a range of about 0.01 to about 10 mmol/min/gram dry cell.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.

The present disclosure provides a method of gaseous substrate fermentation comprising: adding gaseous substrate comprising carbon monoxide into an aqueous medium in a bioreactor; said aqueous medium comprising one or more microorganism; said method comprising measuring cell density; adjusting input of gaseous substrate to increase cell density; changing specific CO uptake in predetermined amounts in a range of about 0.001 to about 10.0 mmol/min/gram dry cell.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising selecting target specific CO uptake; adjusting flow of gaseous substrate such that specific CO uptake equals said target specific CO uptake in a range of about 0.01 to about 10 mmol/min/gram dry cell.

As an embodiment, the present disclosure provides a method gaseous substrate fermentation comprising: adding gaseous substrate comprising carbon monoxide into an aqueous medium in a bioreactor; said aqueous medium comprising one or more microorganism; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.

As embodiments the method of the present disclosure wherein: said microorganism comprises one or more of biologically pure anaerobic acetogenic bacteria; wherein said microorganism comprises one or more of naturally occurring anaerobic acetogenic bacteria; wherein said microorganism comprises one or more of non-naturally occurring anaerobic acetogenic bacteria; wherein said microorganism comprises one or more of non-naturally occurring anaerobic acetogenic bacteria produced by genetic modification using anaerobic acetogenic bacteria as host organism; wherein said microorganism comprises one or more of non-naturally occurring anaerobic acetogenic bacteria produced by inserting genes of anaerobic acetogenic bacteria into a host organism.

As an embodiment the method of the present disclosure wherein said microorganism comprises one or more bacteria selected from Acetogenium kivui, Acetobacterium woodii, Acetoanaerobium noterae, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium autoethanogenum (DSM 23693), Clostridium autoethanogenum (DSM 19630 of DSMZ Germany), Clostridium autoethanogenum (DSM 10061 of DSMZ Germany), Clostridium thermoaceticum, Eubacterium limosum, Clostridium ljungdahlii PETC (ATCC 49587), Clostridium ljungdahlii ER12 (ATCC 55380), Clostridium ljungdahlii C-01 (ATCC 55988), Clostridium ljungdahlii O-52 (ATCC 55889), Clostridium ultunense, Clostridium ragsdali P11 (ATCC BAA-622), Alkalibaculum bacchi CP11 (ATCC BAA-1772), Clostridium coskatii, Clostridium carboxidivorans P7 (ATCC PTA-7827), Geobacter sulfurreducens, Morrella thermacetica, Peptostreptococcus productus, Clostridium drakei, recombinant microorganism (DSM 24138), and mixtures thereof.

As an embodiment the method of the present disclosure wherein said microorganism comprises one or more strains of Clostridium ljundahlii, or one or more strains of Clostridium ragsdalei, or one or more strains of Clostridium carboxidivorans, or one or more strains of Clostridium autoethanogenum.

As an embodiment the method of the present disclosure wherein said microorganism comprises one or more genetically modified microorganism produced by inserting one or more selected genes into host organism selected from any Clostridium ljundahlii strains, or any Clostridium ragsdalei strains, or any Clostridium carboxidivorans strains, or any Clostridium autoethanogenum strains.

As an embodiment the method of the present disclosure wherein said microorganism comprises one or more genetically modified microorganism produced by inserting into any host organism one or more genes from any Clostridium ljundahlii strain, or any Clostridium ragsdalei strain, or any Clostridium carboxidivorans strain, or any Clostridium autoethanogenum strain.

As embodiments the method of the present disclosure: wherein said bioreactor comprises one or more reactor; wherein said bioreactor comprises cell recycle unit; wherein said CO-containing substrate comprises hydrogen.

As an embodiment, further comprising adding nutrient medium to said bioreactor.

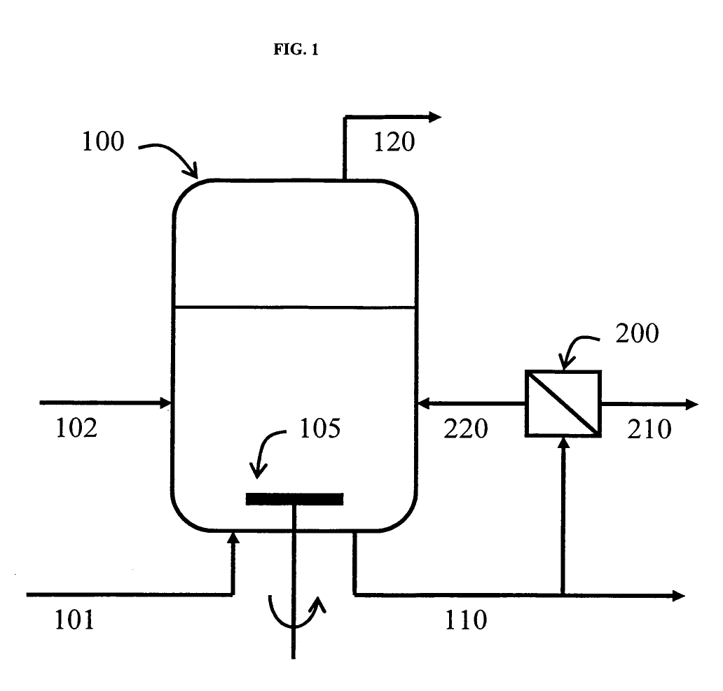

FIG. 1 presents a process for the production of chemical, such as alcohol product mixture, from a gaseous substrate comprising carbon monoxide (CO), such as syngas by fermentation with bacteria, wherein the process comprises a bioreactor (100) containing fermentation broth comprising said bacteria cells and a fermentation medium. A gaseous stream comprising gaseous substrate comprising CO (101) can be fed into the bioreactor along with a stream of fermentation medium (102). A stream of fermentation broth (110) comprising said bacteria cells and said product chemical(s) can be removed from said bioreactor. A stream of fermentor off-gas (120) comprising unused portion of said gaseous stream comprising gaseous substrate is vented from the bioreactor. In one embodiment the stream of fermentor broth (110) flows to a cell recycle apparatus (200) wherein the cells are concentrated and returned (220) to the bioreactor. A permeate stream (210) from said cell recycle apparatus is directed to process of recovery of said chemical(s). In one embodiment at least a portion of the stream of fermentor broth (110) is directed to process of recovery of said alcohol product mixture. In one embodiment at least a portion of the stream of fermentor broth (210) is directed to process of recovery of said alcohol product mixture.

In one embodiment, the bioreactor (100) is equipped with an agitator (105) to provide agitation in order to facilitate contact of gaseous stream comprising gaseous substrate and enhance mass transfer of gaseous substrate with liquid fermentation medium. It is desirable to have good mass transfer rate and thus adequate agitation in the bioreactor throughout the fermentation process.

There are arrangements for collecting samples of gaseous stream comprising gaseous substrate introduced into bioreactor (101) and off-gas leaving bioreactor (120) (not shown in FIG. 1). There is arrangement for collecting samples of fermentation broth of bioreactor (not shown in FIG. 1). Said gas and liquid samples are collected at intervals and analyzed for consumption or production of various gas components, production of various products and the optical density of the fermentation broth.

These measured values can be used to calculate specific carbon monoxide (CO) uptake (SCU) and cell density in fermentation broth in the bioreactor using following equations:

CO uptake,mmol/min=(mmol/min CO input)−(mmol/min CO output) (1)

Cell density,g/L=(Optical density)·(Dilution factor)·(Cell mass constant) (2)

Cell mass,g=(Cell density)·(Volume of bioreactor) (3)

Specific CO uptake,mmol/min/g=(CO uptake)/(Cell mass) (4)

Cell density is mass of cell per unit volume of fermentor broth. Volume of bioreactor is liquid volume in the bioreactor when agitation is turned off. Cell mass constant is mass (g) of dry bacteria cells per liter fermentation broth with optical density of one (1). Optical density is optical density of a sample obtained after dilution of fermentor broth with a suitable solvent such as saline.

Microorganism used in the method of this disclosure may comprise one or more of biologically pure anaerobic acetogenic bacteria.

Microorganism used in the method of this disclosure: may comprise one or more of naturally occurring anaerobic acetogenic bacteria; may comprise one or more of non-naturally occurring anaerobic acetogenic bacteria; may comprise one or more of non-naturally occurring anaerobic acetogenic bacteria produced by genetic modification using anaerobic acetogenic bacteria as host organism; may comprise one or more of non-naturally occurring anaerobic acetogenic bacteria produced by inserting genes of anaerobic acetogenic bacteria into a host organism.

Microorganism used in the method of this disclosure may comprise one or more bacteria selected from Acetogenium kivui, Acetobacterium woodii, Acetoanaerobium noterae, Butyribacterium methylotrophicum, Caldanaerobacter subterraneous, Caldanaerobacter subterraneous pacificus, Carboxydothermus hydrogenoformans, Clostridium aceticum, Clostridium acetobutylicum, Clostridium autoethanogenum (DSM 23693), Clostridium autoethanogenum (DSM 19630 of DSMZ Germany), Clostridium autoethanogenum (DSM 10061 of DSMZ Germany), Clostridium thermoaceticum, Eubacterium limosum, Clostridium ljungdahlii PETC (ATCC 49587), Clostridium ljungdahlii ER12 (ATCC 55380), Clostridium ljungdahlii C-01 (ATCC 55988), Clostridium ljungdahlii O-52 (ATCC 55889), Clostridium ultunense, Clostridium ragsdali P11 (ATCC BAA-622), Alkalibaculum bacchi CP11 (ATCC BAA-1772), Clostridium coskatii, Clostridium carboxidivorans P7 (ATCC PTA-7827), Geobacter sulfurreducens, Morrella thermacetica, Peptostreptococcus productus, Clostridium drakei, recombinant microorganism (DSM 24138), and mixtures thereof.

In one embodiment, microorganism used in the method of this disclosure comprises one or more strains of Clostridium ljundahlii, or one or more strains of Clostridium ragsdalei, or one or more strains of Clostridium carboxidivorans, or one or more strains of Clostridium autoethanogenum.

In one embodiment, microorganism used in the method of this disclosure comprises one or more genetically modified microorganism produced by inserting one or more selected genes into host organism selected from any Clostridium ljundahlii strains, or any Clostridium ragsdalei strains, or any Clostridium carboxidivorans strains, or any Clostridium autoethanogenum strains.

In one embodiment, microorganism used in the method of this disclosure comprises one or more genetically modified microorganism produced by inserting into any host organism one or more genes from any Clostridium ljundahlii strain, or any Clostridium ragsdalei strain, or any Clostridium carboxidivorans strain, or any Clostridium autoethanogenum strain.

In one embodiment of the method of the present disclosure comprises measuring cell density and increasing cell density by adjusting input of gaseous substrate by increasing specific CO uptake. In one embodiment, the method of the present disclosure comprises measuring cell density and increasing cell density by adjusting input of gaseous substrate by decreasing specific CO uptake. In one embodiment, the method of the present disclosure comprises measuring cell density and gradually increasing cell density to desired cell density by adjusting input of gaseous substrate by increasing specific CO uptake. In one embodiment, the method of the present disclosure comprises measuring cell density and gradually increasing cell density to desired cell density by adjusting input of gaseous substrate by decreasing specific CO uptake. In one embodiment, the method of the present disclosure comprises measuring cell density and increasing cell density by gradually increasing specific CO uptake to desired specific CO uptake. In one embodiment, the method of the present disclosure comprises measuring cell density and increasing cell density by gradually decreasing specific CO uptake to desired specific CO uptake. In one embodiment, the method of the present disclosure comprises measuring cell density, increasing cell density and gradually increasing alcohol productivity to desired alcohol productivity by increasing specific CO uptake. In one embodiment, the method of the present disclosure comprises measuring cell density, increasing cell density and gradually increasing alcohol productivity to desired alcohol productivity by increasing specific CO uptake. In one embodiment, the change of specific CO uptake comprises increases in steps of predetermined magnitude. In one embodiment, the change of specific CO uptake comprises decreases in steps of predetermined magnitude. In an embodiment, steps of predetermined magnitude comprise equal magnitude steps. In an embodiment, steps of predetermined magnitude comprise unequal magnitude steps.

In one embodiment, if cell density is less than a target cell density, the method comprises selecting a target specific CO uptake and adjusting flow of gaseous stream comprising gaseous substrate until specific CO uptake equals said target specific CO uptake.

In one embodiment, if cell density is less than a target cell density, the method comprises selecting a target specific CO uptake and adjusting flow of gaseous stream comprising gaseous substrate until specific CO uptake equals said target specific CO uptake; and repeating selecting a target specific CO uptake and adjusting flow of gaseous stream comprising gaseous substrate until specific CO uptake equals said target specific CO uptake.

Value of said target specific CO uptake may comprise a range of about 0.1 to about 10.0 mmol CO per minute per gram dry microorganism. Value of said desired specific CO uptake may comprise a range of about 0.1 to about 10 mmol/min/g.

Value of said target cell density may comprise a range of about 0.1 to about 50 g/L. Value of said desired cell density may comprise a range of 0.5 to 50 g/L.

Value of said desired ethanol productivity comprises a range of 1 to 50 g/L/day.

Value of said desired ethanol concentration in the fermentation broth comprises a range of 1 to 20 g/L.

Typically in a laboratory scale bioreactor such as New Brunswick Bioflow I bioreactor, agitator speed in the range of 300-900 revolutions per minute (rpm) provides adequate agitation for desirable mass transfer rate. In one embodiment, agitator speed in the range of 500-700 rpm is used. In one embodiment, agitator speed in the range of 550-650 rpm is used. In one embodiment, agitator speed of about 600 rpm is used.

In an embodiment, for a larger scale bioreactor such as a bioreactor of about 100 to 500 liter size, agitator speed in the range of about 50 to about 500 rpm is used for agitation. In an embodiment, for a commercial scale bioreactor of about 100,000 to about 1000,000 liter size, agitator speed in the range of about 1 to about 50 rpm is used for agitation. In various embodiments, a larger bioreactor requires smaller rpm compared to a smaller bioreactor.

As an embodiment, the present disclosure provides temperature control in the bioreactor in the range of 25 to 50° C.

In one embodiment of the method of the present disclosure, said bioreactor comprises one reactor. In one embodiment of the method of the present disclosure, said bioreactor comprises two or more reactors.

In one embodiment of the method of the present disclosure, said bioreactor comprises cell recycle unit.

In one embodiment of the method of the present disclosure, said gaseous stream comprising gaseous substrate comprising CO also comprises hydrogen. In one embodiment, said gaseous stream comprising gaseous substrate comprising CO comprises syngas. In one embodiment, said gaseous stream comprising gaseous substrate comprising CO comprises steel mill off-gas. In one embodiment, said gaseous stream comprising gaseous substrate comprising CO comprises syngas obtained by gasification of carbonaceous material comprising biomass.

In one embodiment one or more growth or seed fermentors provide the initial supply of inoculum of bacteria cells. In one embodiment one or more growth or seed fermentors continue to supply bacteria cells to bioreactor in conjunction with the method of this disclosure. In one embodiment of the present disclosure, the process comprises cell recycle.

A method of producing an alcohol product mixture comprising:

adding gaseous substrate comprising carbon monoxide into an aqueous medium in a bioreactor; said aqueous medium comprising one or more microorganism; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired specific CO uptake in a range of about 0.01 to about 10 mmol/min/gram dry cell; further comprising adding continuous flow of aqueous medium into bioreactor; removing a continuous flow of fermentation broth from the bioreactor.

A method of producing an alcohol product mixture comprising:

adding gaseous substrate comprising carbon monoxide into an aqueous medium in a bioreactor; said aqueous medium comprising one or more microorganism; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until obtaining a desired ethanol productivity a range of about 1 to about 50 g/L; further comprising adding continuous flow of aqueous medium into bioreactor; removing a continuous flow of fermentation broth from the bioreactor.

A method of producing an alcohol product mixture comprising:

adding gaseous substrate comprising carbon monoxide into an aqueous medium in a bioreactor; said aqueous medium comprising one or more microorganism; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining a desired ethanol concentration in the fermentation broth a range of about 1 to about 50 g/L; further comprising adding continuous flow of aqueous medium into bioreactor; removing a continuous flow of fermentation broth from the bioreactor.

It is envisioned that the method of the present disclosure embodies: a batch process, a semi-batch process, a fed batch process, transition from to a continuous process, continuous process.

The present disclosure provides a method of gaseous substrate fermentation comprising: adding gaseous substrate comprising carbon monoxide (CO) into an aqueous medium in a bioreactor; said aqueous medium comprising one or more microorganism; said method comprising measuring cell density; measuring rate of input of CO and rate of output of CO; determining specific CO uptake; adjusting rate of input of CO to increase cell density in a way that specific CO uptake changes in predetermined amounts; optionally repeating these actions.

Nutrient medium comprises microorganism growth medium which may contain one or more of vitamins and minerals that permit growth of selected microorganism. Table 1 provides an embodiment of nutrient medium as contemplated by the present disclosure. Other nutrient medium suitable for the present disclosure is known in the art. Moreover, nutrient medium that is not disclosed in the art but derived from various components described in Table 1 can be utilized by the present invention. The present disclosure provides for improved compositions of nutrient medium.

TABLE 1
Medium Component and Their Concentrations

| Component/ | | |
|---|---|---|
| Ion | Added As | Conc in ppm |
| NH₄⁺ | NH₄Cl/ | ≦838 |
| | (NH₄)₂HPO₄ | |
| Fe | FeCl₂•4H₂O | ≦17 |
| Ni | NiCl₂•6H₂O | ≦0.2 |
| Co | CoCl₂•6H₂O | ≦1.0 |
| Se | Na₂SeO₃ | ≦0.1 |
| Zn | ZnSO₄•7H₂O | ≦0.5 |
| Mo | Na₂MoO₄•2H₂O | ≦0.3 |
| Mn | MnCl₂•4H₂O | ≦0.2 |
| B | H₃BO₃ | ≦1.1 |
| Cu | CuCl₂•2H₂O | ≦0.15 |
| W | Na₂WO₄•2H₂O | ≦1.2 |
| K | KCl | ≦79 |
| Mg | MgCl₂•6H₂O | ≦60 |
| Na | NaCl | ≦80* |
| Ca | CaCl₂•2H₂O | ≦55 |
| Cysteine HCl | Cysteine HCl | ≦250 |
| PO₄⁻² | H₃PO₄/ | ≦820 |
| | (NH₄)₂HPO₄ | |
| Pantothenic | Pantothenic Acid | ≦0.04 |
| Acid | | |
| Biotin | Biotin | ≦0.02 |
| Thiamin | Thiamine | ≦0.05 |

*Na⁺ concentration is from NaCl only. It does not include Na⁺ from the other components such as Na₂WO₄•2H₂O.
** Ca⁺² concentration does not include calcium from pantothenic acid, calcium salt (i.e. Calcium d-Pantothenate).

### EXAMPLES

Comparative Example (See Example 11 in U.S. Pat. No. 7,285,402)

To prepare the stock cultures for inoculation of the reactor, cultures of Clostridium ljungdahlii, strain C-01 (ATCC Accession No. 55988) were grown up in 150 mL serum bottles on CO, CO₂ and H₂ in a rich medium containing 1 g/L yeast extract and 1 g/L trypticase, in salts and vitamins. The vitamin concentration employed was 0.4 mL/L medium of an aqueous solution containing 50.5 mg/L calcium pantothenate, 20.6 mg/L d-biotin and 50.6 mg/L thiamine HCl. Bottles were incubated at 37° C. in a shaker incubator. The cultures were grown to the exponential growth phase, as determined by visual inspection. With each inoculation, approximately 90 mL of stock culture were transferred from serum bottles to 1 liter of medium, representing 9% by volume inoculation. A successful inoculation is described below. The outlined procedure can be repeated several times to obtain a successful inoculation.

In obtaining a successful inoculation, 90 mL/L of inoculum were added to a 1 liter batch of basal medium containing 0.4 mUL vitamins and salts (t=0). The agitation rate was 240 rpm, the pH was 5.3, the temperature was 38.5° C. and the gas retention time (continuous gas flow) was 110 minutes. The gas feed contained 62% H₂, 31% CO and 7% C₂H₆. After 13 hr (t=13 hr) some CO conversion was noted, and at t=23 hr the agitation rate was increased from 240 rpm to 300 rpm. The gas retention time was decreased to 100 minutes at t=27 hr, and a further decrease in gas retention time was made at t=46 hr. The agitation rate was also increased in 100 rpm increments at t=28 hr, 59 hr, 72 hr and 85 hr.

By t=110 hr, the system was operating with a gas retention time of 80 minutes and an agitation rate of 600 rpm. The cell concentration was 0.5 g/L and the CO conversion was 35%. There was still no H₂ conversion, but small amounts of ethanol and acetate (about. 1 g/L each) had accumulated in the batch culture broth. The efforts up until this time emphasized cell growth in the reactor.

Medium flow using the same concentrations as in basal medium was started at a rate of 0.4 ml/min at t=120 hr. A program of nominal increases in gas rate, agitation rate and medium rate was then initiated while carefully maintaining the system under excess H₂. By t=210 hr, the ethanol concentration was 17 g/L, the acetate concentration was 1 g/L, the cell concentration was 1.6 g/L, the CO conversion was nearly 100% and the H₂ conversion was 90%. The ethanol productivity reached 11.4 g/L-day.

A program of gradual gas rate increases was again started. Concurrent vitamin increases were made to bring the vitamin addition rate to 0.7 ml/L medium. By t=610 hr, the reactor was producing 20 g/L ethanol and about 2 g/L acetate. The CO conversion was nearly 100% and the H₂ conversion was 85%. The ethanol productivity reached 14 g/Lday.

Fermentation Medium for examples 1-5 comprise one or more components selected from those presented in Table 1.

### Example 1

### Clostridium ljungdahlii PETC; Starting-Up a Reactor Using Specific Carbon Monoxide as a Guide to Increase Gas

New Brunswick bioflow I reactor containing Fermentation Medium was started with 0.32 g/L of actively growing Clostridium ljungdahlii PETC strain. The rate of agitation of the reactor was set to 600 rpm at the start of the experiment. This agitation rate was maintained throughout the experiment. Temperature in the bioreactor was maintained in the range of about 38.5 to about 39° C. throughout the experiment. Samples of syngas feed into the bioreactor and off-gas from the bioreactor and fermentation broth in the bioreactor were taken at intervals, for example feed gas, off-gas and fermentation broth was sampled about daily, once two hours and once four hours respectively. Above samples were analyzed for consumption or production of various gas components, broth acetic acid concentration, broth ethanol concentration and the optical density (cell density) of the culture. The unaroused volume of the reactor was maintained between 1300 to 1400 mL throughout the experiment.

Thus specific CO uptake (SCU) was determined using equations (1)-(4) described above.

In this particular example targeted SCU value was set between 0.75 and 0.89 mmol/min/g (of cells). Also the rate of gas flow to the reactor was not increased if the culture was not utilizing 80% of the CO provided to the reactor at a given point.

In this particular experiment a cell recycle system (CRS) was attached to the reactor before the start of the experiment. 18.6 hours after the inoculation 60 mL of growth medium was directly added to the medium and then media flow to the reactor was started at 0.41 mL/min. 21.5 hours after the inoculation the rate of flow of nutrients to the reactor was increased to 1.1 mL/min and through the cell recycle system 1 mL/min permeate was drawn out from the reactor. The above step was taken to prevent accumulation of inhibitory amounts of acetic acid and ethanol in the culture and also to provide adequate amounts of nutrients to the culture.

Cell density of the reactor increased with time and reached 3 g/L of cells within 45 hours after the inoculation of the reactor. At this point culture was producing more than 6 g/L of ethanol and about 8 g/L acetic acid.

In this particular experiment pH of the culture was maintained between 4.69 and 4.71 through out the experiment.

After bacteria started growing actively in the reactor (when the cell density of the reactor reach about 50% more than the initial cell density) culture was supplemented with composition of vitamins (in addition to the vitamins already in the medium) if the acetic acid concentration of the culture broth is below a predetermined value. Criteria used to add cocktail of vitamins to the culture was as follow as: if the culture broth acetic acid is less than about 2.5 g/L, about 0.34 mL of vitamins per liter of culture was added, if the culture broth acetic acid is less than about 2 g/L, about 0.67 mL of vitamins per liter of culture was added, if the culture broth acetic acid is less than about 1.5 g/L, about 1 mL of vitamins per liter was added. Composition of vitamins used in these experiments were as follows:

| | |
|---|---|
| Biotin | 0.08-1 μM |
| Thiamin HCl | 0.12-1.5 μM |
| Calcium d-pantothenate | 0.15-2 μM |

ATCC vitamin supplement (catalog No. MD-VS) was added to PETC example to the final concentration of 1% (of fermentation medium) in addition to the Biotin, Thiamin and calcium pantothenate.

### Example 2

### Clostridium ljungdahlii C-01

New Brunswick Bioflow I bioreactor containing about 1.5 liter (e.g. in the range of 1.45 to 1.65 liters) of Fermentation Medium was started with about 0.3 g/L of actively growing Clostridium ljungdahlii C-01 strain. At the start of the experiment, the rate of agitation in the bioreactor was set to 600 rpm. This agitation rate was maintained throughout the experiment. Temperature in the bioreactor was maintained in the range of about 36 to about 37.5° C. throughout the experiment. Samples of the following were taken and analyzed at different intervals (e.g. 1 to 4 hour interval): syngas feed into the bioreactor; off-gas from the bioreactor; fermentation broth in the bioreactor. The sample analysis provided: consumption of various gaseous components, production of various gaseous components, concentration of acetic acid, concentration of ethanol and optical density of the fermentation broth.

Thus specific CO uptake (SCU) was determined using equations (1)-(4) described above.

Initially, a value of syngas input was calculated using above equations corresponding to SCU value of about 1.4 mmol/min/g and flow of syngas was maintained at this calculated value until cell density increased and reached a value of about 1.5 g/L.

Once the cell density of the reactor reached about 1.5 g/L the set SCU value to predict gas was lowered to about 1.2 mmol/min/g. Thereafter once the cell mass of the reactor reached about 2.5 g/L the set SCU value to predict gas was lowered to about 1.0 mmol/min/g. Cell mass increased with time and reached the expected cell mass of about 2.8 g/L within about 79 hours after the inoculation of the reactor. At this point culture was producing more than about 20 g/L of ethanol.

About 13.97 hours after the inoculation media flow to the reactor was started at about 0.2 ml/min (approximate cell retention time: about 125 hours). About 28.08 hours after the inoculation media flow to the reactor was increased to about 0.5 ml/min (approximate cell retention time: about 52 hours). During the experiment pH was maintained around about 4.5.

The gradual lowering of the set SCU through out the start-up procedure is to facilitate the gradual transformation of the culture to low SCU (between about 0.7 to about 0.9 mmol/min/g) maintain during the production mode (steady state) of the reactor.

Above mention process takes less than about 80 hours to reach the set goal of cell mass (about 2.8 g/L) of the reactor.

### Example 3

### Clostridium autoethanogenum

New Brunswick Bioflow I bioreactor containing about 1.5 liter (e.g. in the range of about 1.45 to about 1.65 liters) of Fermentation Medium was started with about 0.47 g/L of actively growing Clostridium autoethanogenum. At the start of the experiment, the rate of agitation in the bioreactor was set to about 600 rpm. This agitation rate was maintained throughout the experiment. Temperature in the bioreactor was maintained in the range of about 36 to about 37.5° C. throughout the experiment. Samples of the following were taken and analyzed at different intervals (e.g. about 1 to about 4 hour interval): syngas feed into the bioreactor; off-gas from the bioreactor; fermentation broth in the bioreactor. The sample analysis provided: consumption of various gaseous components, production of various gaseous components, concentration of acetic acid, concentration of ethanol and optical density of the fermentation broth.

Thus specific CO uptake (SCU) was determined using equations (1)-(4) described above.

Initially, a value of syngas input was calculated using above equations corresponding to SCU value of about 0.4 mmol/min/g and flow of syngas was maintained at this calculated value until cell density increased. Gas flow corresponding to target SCU value of about 0.4 mmol/min/g was maintained for about 19 hours. Between the period of 19 and 21 hours after the inoculation targeted SCU value was about 0.5 mmol/min/g. Targeted SCU value was set to about 0.6 at about 21 hours after the inoculation. Cell density increased with time and reached about 3 g/L within about 79 hours after the inoculation of the reactor. At this point culture was producing more than about 15 g/L of ethanol. About 26 hours after the inoculation media flow to the reactor was started at about 0.1 ml/min (approximate cell retention time: about 240 hours). About 50 hours after the inoculation media flow to the reactor was increased to about 0.2 ml/min (approximate cell retention time: about 119 hours). About 71 hours after the inoculation media flow to the reactor was increased to about 0.5 ml/min (approximate cell retention time: about 50 hours). During the experiment pH was maintained around about 4.5.

### Example 4

### Clostridium autoethanogenum

New Brunswick Bioflow I bioreactor containing about 1.5 liter (e.g. in the range of about 1.45 to about 1.65 liters) of Fermentation Medium was started with about 0.47 g/L of actively growing Clostridium autoethanogenum. At the start of the experiment the rate of agitation in the bioreactor was set to about 600 rpm. This agitation rate was maintained throughout the experiment. Temperature in the bioreactor was maintained in the range of about 36 to about 37.5° C. throughout the experiment. Samples of the following were taken and analyzed at different intervals (e.g. 1 to 4 hour interval): syngas feed into the bioreactor; off-gas from the bioreactor; fermentation broth in the bioreactor. The sample analysis provided: consumption of various gaseous components, production of various gaseous components, concentration of acetic acid, concentration of ethanol and optical density of the fermentation broth.

Thus specific CO uptake (SCU) was determined using equations (1)-(4) described above.

Initially, a value of syngas input was calculated using above equations corresponding to SCU value of about 0.6 mmol/min/g and flow of syngas was maintained at this calculated value until cell density increased. Targeted SCU value was set to about 0.7 at about 26 hours after the inoculation. Cell mass increased with time and reached about 2.97 g/L of cell mass within 64 hours after the inoculation of the reactor. At this point culture was producing more than about 18 g/L of ethanol. About 18.3 hours after the inoculation media flow to the reactor was started at about 0.1 ml/min (approximate cell retention time: about 242 hours). About 41.6 hours after the inoculation media flow to the reactor was increased to about 0.2 ml/min (approximate cell retention time: about 121 hours). During the experiment pH was maintained around about 4.5.

### Example 5

### Butyribacterium methylotrophicum (ATCC 33266); Starting-Up a Reactor Using Specific Carbon Monoxide as a Guide to Increase Gas

In this experiment specific carbon monoxide uptake start-up method was tested with a non-clostridial acetogen.

This experiment was started in a New Brunswick bioflow I reactor containing 1.31 g/L of actively growing Butyribacterium Methylotrophicum in the previously mentioned fermentation medium. The rate of agitation of the reactor was set to 700 rpm at the start of the experiment. This agitation rate was maintained throughout the experiment. Temperature in the bioreactor was maintained between 38.5 to 38.6° C. throughout the experiment.

Samples of syngas feed into the bioreactor and off-gas from the bioreactor and fermentation broth in the bioreactor were taken at intervals, for example feed gas, off-gas and fermentation broth was sampled about daily, once two hours and once four hours respectively. Above samples were analyzed for consumption or production of various gas components, broth acetic acid concentration, broth ethanol concentration and the optical density (cell density) of the culture. The unaroused volume of the reactor was maintained between 1150 to 1100 mL throughout the experiment.

Thus specific CO uptake (SCU) was determined using equations (1)-(4) described above.

In this particular example targeted SCU value was set to 0.8 mmol/min/g (of cells).

To maintain the stability of the culture the rate of gas flow to the reactor was not increased if the culture was not utilizing 80% of the CO provided to the reactor at any given point.

In this experiment a Cell Recycle System (CRS) was attached to the reactor before the start of the experiment. Fermentation medium (nutrient) flow to the reactor was started at a rate of 1 mL/min and through the CRS 0.9 mL/min permeate was drawn out from the reactor.

Cell density of the reactor increased with time and reached 5.27 g/L of cells within 24 hours after the inoculation of the reactor. At this point culture was producing more than 15 g/L of ethanol, 0.3 g/L butanol, and more than 2 g/L acetic acid.

In this particular experiment pH of the culture was maintained between 4.67 and 4.71 through out the experiment.

Numerous modifications and variations of the present disclosure could be made by those skilled in the art without departing from the scope of the present disclosure included in the specific embodiments, examples, claims, application, etc., thereof. All published documents are incorporated by reference herein.

We claim:

1. A process of producing one or more alcohols from a gaseous substrate, comprising: fermenting a gaseous substrate comprising carbon monoxide (CO) into an aqueous medium in a bioreactor; said process comprising increasing cell density by adjusting specific CO uptake in said aqueous medium.

2. The method of claim 1 wherein rate of change of specific CO uptake comprises controlling rate of input of CO.

3. The method of claim 1 wherein rate of change of specific CO uptake comprises measuring rate of CO input; measuring rate of CO output; and measuring cell mass.

4. The method of claim 1, further comprising changing specific CO uptake in predetermined amounts.

5. The method of claim 4, wherein the said predetermined amounts comprise a range of about 0.001 to about 10.0 mmol/min/gram dry cell.

6. The method of claim 4, wherein the said predetermined amounts comprise a range of about 0.01 to about 5.0 mmol/min/gram dry cell.

7. The method of claim 4, wherein the said predetermined amounts comprise a range of about 0.1 to about 1.0 mmol/min/gram dry cell.

8. The method of claim 1, further comprising adding a flow of aqueous medium into bioreactor; removing a flow of fermentation broth from the bioreactor.

9. The method of claim 1, further comprising adding a continuous flow of aqueous medium into bioreactor; removing a continuous flow of fermentation broth from the bioreactor.

10. A method gaseous substrate fermentation comprising:
adding gaseous substrate comprising carbon monoxide into an aqueous medium in a bioreactor; said method comprising selecting target specific CO uptake; adjusting flow of gaseous substrate such that specific CO uptake equals said target specific CO uptake in a range of about 0.01 to about 10 mmol/min/gram dry cell.

11. A method gaseous substrate fermentation comprising:
adding gaseous substrate comprising carbon monoxide into an aqueous medium in a bioreactor; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.

12. The method of claim 1 wherein said aqueous medium comprises one or more of the microorganism including: biologically pure microorganism, naturally occurring microorganism, non-naturally occurring microorganism, non-naturally occurring microorganism produced by genetic modification, mutant of naturally occurring microorganism, mutant of non-naturally occurring microorganism, recombinant microorganism, engineered microorganism, artificially synthesized microorganism.

13. The method of claim 1 wherein said bioreactor comprises one or more reactor.

14. The method of claim 1 wherein said bioreactor comprises cell recycle unit.

15. The method of claim 1 wherein said CO-containing substrate comprises hydrogen.

16. The method of claim 1 further comprising adding nutrient medium to said bioreactor.

17. A method gaseous substrate fermentation comprising:
adding gaseous substrate comprising carbon monoxide into an aqueous medium in a bioreactor; said method comprising selecting target specific CO uptake; adjusting flow of gaseous substrate such that specific CO uptake equals said target specific CO uptake in a range of about 0.01 to about 10 mmol/min/gram dry cell.

18. A method gaseous substrate fermentation comprising:
adding gaseous substrate comprising carbon monoxide into an aqueous medium in a bioreactor; said method comprising the steps of selecting target specific CO uptake in a range of about 0.01 to about 10 mmol/min/gram dry cell, adjusting flow of gaseous substrate such that specific CO uptake equals said target specific CO uptake; repeating said steps until attaining desired cell density in a range of about 0.1 to about 15 g/L; upon attaining said desired cell density converting to continuous mode of operation.
